(19) United States
(12) Patent Application Publication
(10) Pub. No.: US 2011/0082565 A1
(43) Pub. Date: Apr. 7, 2011

(54) NONWOVEN STRUCTURE AND METHOD OF FABRICATING THE SAME

(75) Inventors: Roey Tzezana, Haifa (IL); Shulamit Levenberg, Moreshet (IL); Eyal Zussman, Haifa (IL)

(73) Assignee: TECHNION RESEARCH & DEVELOPMENT

(21) Appl. No.: 12/996,776

(22) PCT Filed: Jun. 8, 2009
(86) PCT No.: PCT/IL09/00571

§ 371 Date: Dec. 8, 2010

Related U.S. Application Data
(60) Provisional application No. 61/060,285, filed on Jun. 10, 2008.

(51) Int. Cl. A61F 2/02; B32B 7/02; D04H 3/02; B32B 37/16

(52) U.S. Cl. 623/23.72; 428/220; 156/60; 156/273.1

(57) ABSTRACT

A method for fabricating a nonwoven structure is disclosed. The method comprises: forming a nonwoven layer of polymer fibers on the collector liquid surface, transferring the layer to a solid surface, and repeating the formation and the transfer in a layerwise manner. The method is generally effected by spinning liquefied polymer, in particular by employing electrostatic or hydrostatic forces. The nonwoven structure formed comprises of plurality of nonwoven layers: the average thickness of the nonwoven structure is at least 0.5 mm and the structure has an overall porosity of at least 90%. The nonwoven structure an be used in forming a scaffold for tissue engineering, in particular a cell-scaffold composition, suitable for implanting in a patient, wherein at least one cell population is seeded on at least one of the plurality of layers.

### RELATED APPLICATION/S

This application claims the benefit of priority from U.S. Patent Application No. 61/060,285, the contents of which are hereby incorporated by reference as if fully set forth herein.

### FIELD AND BACKGROUND OF THE INVENTION

The present invention, in some embodiments thereof, relates to a nonwoven structure and, more particularly, but not exclusively, to a multilayered nonwoven structure. Some embodiments of the present invention relate to a method for fabricating the multilayered nonwoven structure and various uses thereof.

Fabrication of nonwoven articles having nanometric and micrometric internal structure receives increasing attention due to the potential application of such articles in many fields, particularly in the field of medicine, e.g., tissue engineering.

Tissue engineering, particularly tissue regeneration, repair and/or implant are used in treating damaged, traumatized, abnormal functioning, diseased and/or dysfunction tissues. Tissue repair and/or regeneration are based on transplanting scaffolds, membranes or matrices along with cells which are capable of growing into and repairing damaged or diseased tissues. These scaffolds serve as temporary artificial extracellular matrices, accommodating cells and supporting three-dimensional tissue regeneration. Desired scaffolds, membranes or matrices for tissue regeneration are biocompatible and/or biodegradable materials capable of supporting the growth and/or regeneration of soft or hard tissues. Such substances should therefore be compatible with the desired cure.

Various applications utilizing scaffolds for tissue engineering have been previously described, some are summarized infra.

PCT Publication No. WO06036826 discloses tissue engineering scaffolds for in vitro and in vivo use. These scaffolds comprise a nanofibrous, nanoporous hydrogel formed from self-assembling peptides. The peptides comprising the hydrogel may be biodegradable materials, include ceramics (e.g. hydroxylapatite), biodegradable polymers, including polycaprolactone (PCL) and polylactic acid (PLA), or non-biodegradable materials (e.g. silk). Furthermore, these scaffolds comprise cells (e.g. stem cells, progenitor cells).

U.S. Pat. No. 7,122,057 discloses use of engineered regenerative biostructures (ERB) as a bone substitute. These biostructures comprise ceramic materials (e.g., hydroxylapatite) that are partially joined to each other in a manner that leaves some porosity therebetween. According to U.S. Pat. No. 7,122,057, the micro- and meso-architecture of the ERBs is designed to be consistent and defined. Furthermore, the ERBs may comprise cells (e.g. MSCs) or polymers (e.g. PLA and PCL).

Another approach for designing porous scaffolds for use in tissue engineering is electrospinning [Reneker et al., Advances in applied mechanics 2007, 41(43), Rutledge et al., Adv Drug Deliv Rev 2007, 59(14)]

In electrospinning, a fine stream or jet of liquid is produced by pulling a small amount of charged liquefied polymer through space using electrical forces. The produced fibers are hardened and collected on a suitably located precipitation device to form a nonwoven article. In the case of a liquefied polymer which is normally solid at room temperature, the hardening procedure may be mere cooling, however other procedures such as chemical hardening or evaporation of solvent may also be employed.

Electrospinning yields scaffolds with a high porosity. The nanometer to micrometer fibers comprised therein combine in a nonwoven structure resembling the natural extracellular matrix. In the electrospinning process, many parameters can be altered to optimize the properties of the final product. Cells growing on electrospun scaffolds have been shown to exhibit higher attachment and proliferation than cells growing on a smooth or scaffolds made of thicker fibers. Generally, nanoscale architectures cause the cell to create more filopodia and add to the proliferation and attachment ability of the cell [Mikos et al., Tissue Engineering 2006, 12(5)].

Several types of electrospun scaffolds are also disclosed in U.S. Published Application Nos. 20060204539, 20070269481, 20060067969 and 20060263417.

### SUMMARY OF THE INVENTION

According to an aspect of some embodiments of the present invention there is provided a method of fabricating a nonwoven structure. The method comprises: forming a nonwoven layer of polymer fibers on the collector liquid surface; transferring the layer to a solid surface; and repeating the formation and the transfer in a layerwise manner thereby forming a nonwoven multilayer structure on the solid surface.

According to some embodiments of the invention the nonwoven layer is formed by spinning liquefied polymer.

According to some embodiments of the invention the liquefied polymer is charged and the spinning is effected by electrostatic forces.

According to some embodiments of the invention the spinning is effected by hydrostatic forces.

According to some embodiments of the invention the method further comprises drying the multilayer structure, e.g., by vacuum desiccation.

According to some embodiments of the invention the collector liquid is an aqueous liquid.

According to some embodiments of the invention the collector liquid is an organic solvent.

According to some embodiments of the invention the collector liquid comprises saline.

According to some embodiments of the invention the method further comprises seeding cells on at least one layer of the multilayer structure.

According to some embodiments of the invention the seeding is applied to an outermost layer of the multilayer structure.

According to some embodiments of the invention the seeding is applied directly to at least one internal layer of the multilayer structure.

According to some embodiments of the invention the seeding is applied in a layerwise manner to a plurality of layers of the multilayer structure.

According to some embodiments of the invention the seeding is applied such as to form a predetermined density distribution of cells along a thickness direction of the multilayer structure.

According to some embodiments of the invention the method further comprises incorporating at least one agent in the multilayer structure.

According to some embodiments of the invention the multilayer structure is formed at a rate of at least 20 cubic millimeters per minute.

According to an aspect of some embodiments of the present invention there is provided a nonwoven structure. The structure comprises a plurality of nonwoven layers of fibers, wherein an average thickness of the nonwoven structure is at least 0.5 mm, and wherein an overall porosity of the nonwoven structure is at least 90%.

According to some embodiments of the invention at least two adjacent layers of the plurality of layers are at least partially spaced apart such the at least two adjacent layers are distinguishable from one another solely by the at least partial spacing.

According to some embodiments of the invention the plurality of layers comprises at least 50 layers.

According to some embodiments of the invention an average thickness per layer is at least 10 times an average fiber diameter.

According to an aspect of some embodiments of the present invention there is provided a scaffold for tissue engineering. The scaffold comprises the nonwoven structure described herein.

According to an aspect of some embodiments of the present invention there is provided a cell-scaffold composition. The composition comprises the scaffold described herein and at least one cell population seeded on at least one of the plurality of layers.

According to some embodiments of the invention the cell population has a predetermined density distribution along a thickness direction of the multilayer structure.

According to some embodiments of the invention an average thickness of the multilayer structure is at least 1 cm and the cell population occupies the multilayer structure at any depth within the multilayer structure.

According to an aspect of some embodiments of the present invention there is provided a method of treating a patient. The method comprises: implanting in the patient a cell-scaffold composition which comprises a multilayered non woven scaffold and at least one cell population seeded on at least one of the plurality of layers, wherein the scaffold has an average thickness of at least 0.5 mm, and an overall porosity of at least 90%.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C are schematic illustrations of a nonwoven structure, according to various exemplary embodiments of the present invention;

FIG. 2 is a flowchart diagram describing a method suitable for fabrication a nonwoven structure, according to various exemplary embodiments of the present invention;

FIG. 3 is a flowchart diagram describing a method suitable for fabrication a seeded nonwoven structure, in embodiment of the invention in which cells are seeded on an internal layer of the structure;

FIG. 4 is a flowchart diagram describing a method suitable for treating a patient, according to various exemplary embodiments of the present invention;

FIG. 5 is a schematic illustration of a system for spinning polymer fibers on a collector liquid surface, according to various exemplary embodiments of the present invention;

FIGS. 6A-E illustrate a multilayer structure fabrication process as executed in experiments performed according to various exemplary embodiments of the present invention;

FIGS. 7A-C are scanning electron microscopy (SEM) images showing cross-sections of a multilayer scaffolds prepared according to various exemplary embodiments of the present invention.

FIG. 7D is a SEM image showing cross-sections of a control multilayer scaffold, prepared in experiment performed by the present inventors;

FIG. 7E is a SEM image of hydrospun fibers of nanometric size fabricated according to some embodiments of the present invention;

FIG. 7F is a SEM image of electrospun fibers of nanometric size fabricated in experiment performed by the present inventors;

FIG. 8A shows SEM images of a scaffold as prepared according to various exemplary embodiments of the present invention (left) and a control scaffold (right);

FIG. 8B shows the scaffold of FIG. 8A left, cut open to reveal its multilayer structure;

FIG. 9A shows a scaffold having micrometric fibers, prepared according to various exemplary embodiments of the present invention and seeded with myoblasts and stained with anti-desmin (red), phalloidin (green) and DAPl (blue);

FIG. 9B shows a control scaffold having micrometric fibers, seeded with myoblasts and stained with anti-desmin (red), phalloidin (green) and DAPl (blue);

FIG. 9C shows a scaffold having micrometric fibers, prepared according to various exemplary embodiments of the present invention and seeded with hESCs and stained with anti-vimentin (red), phalloidin (green) and DAPl (blue);

FIG. 9D shows a control scaffold having micrometric fibers, seeded with hESCs and stained with anti-vimentin (red), phalloidin (green) and DAPl (blue);

FIG. 10A shows control scaffold having nanometric fibers and seeded with myoblasts stained with anti-desmin (red), phalloidin (green) and DAPl (blue);

FIGS. 10B and 10C show scaffold having nanometric fibers, prepared according to various exemplary embodiments of the present invention and seeded with myoblasts stained with anti-desmin (red), phalloidin (green) and DAPl (blue);

FIG. 10D shows a control scaffold having nanometric fibers and seeded with hESCs stained with anti-desmin (red), phalloidin (green) and DAPl (blue);

FIG. 10E shows scaffold having nanometric fibers prepared according to various exemplary embodiments of the present invention and seeded with hESCs stained with anti-desmin (red), phalloidin (green) and DAPl (blue);

FIG. 10F shows an inner pore filled with cells inside a scaffold having nanometric fibers which was prepared according to various exemplary embodiments of the present invention;

FIG. 10G shows a control scaffold having micrometric fibers and seeded with myoblasts stained with anti-vimentin (red), phalloidin (green) and DAPI (blue);

FIG. 10H shows scaffold having micrometric fibers prepared according to various exemplary embodiments of the present invention and seeded with myoblasts stained with anti-vimentin (red), phalloidin (green) and DAPI (blue); and

FIGS. 11A and 11B show a cross-section of a scaffold having micrometric fibers, 4 days after the scaffold was seeded with myoblasts via a layerwise seeding technique according to various exemplary embodiments of the present invention.

### DESCRIPTION OF SPECIFIC EMBODIMENTS OF THE INVENTION

The present invention, in some embodiments thereof, relates to a nonwoven structure and, more particularly, but not exclusively, to a multilayered nonwoven structure. Some embodiments of the present invention relate to a method for fabricating the multilayered nonwoven structure and various uses thereof.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

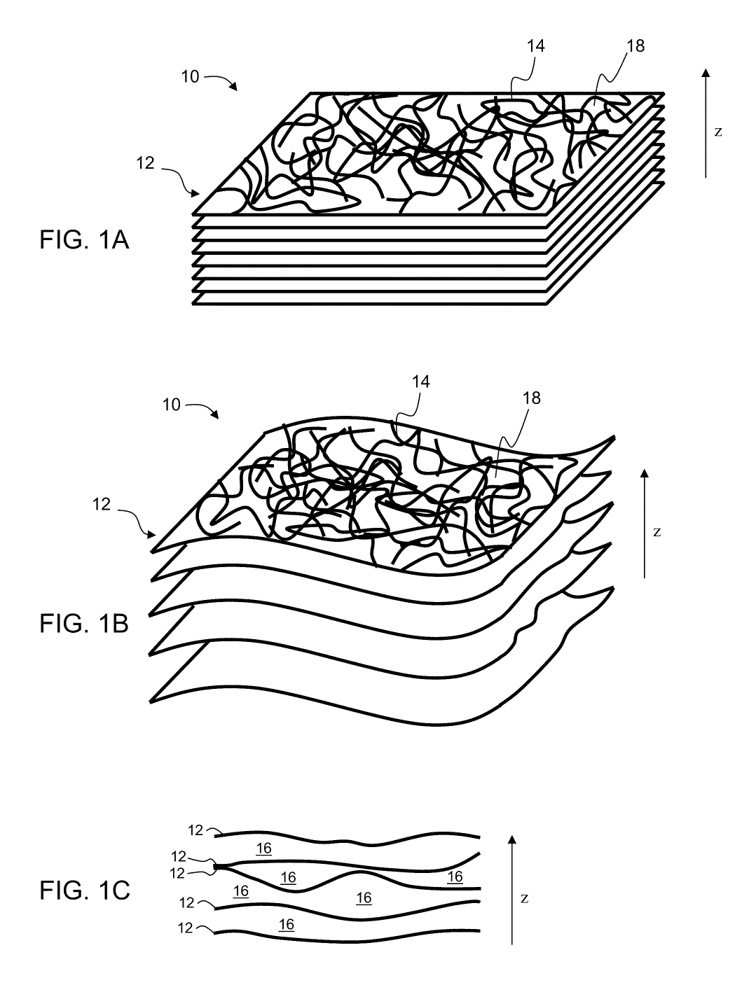

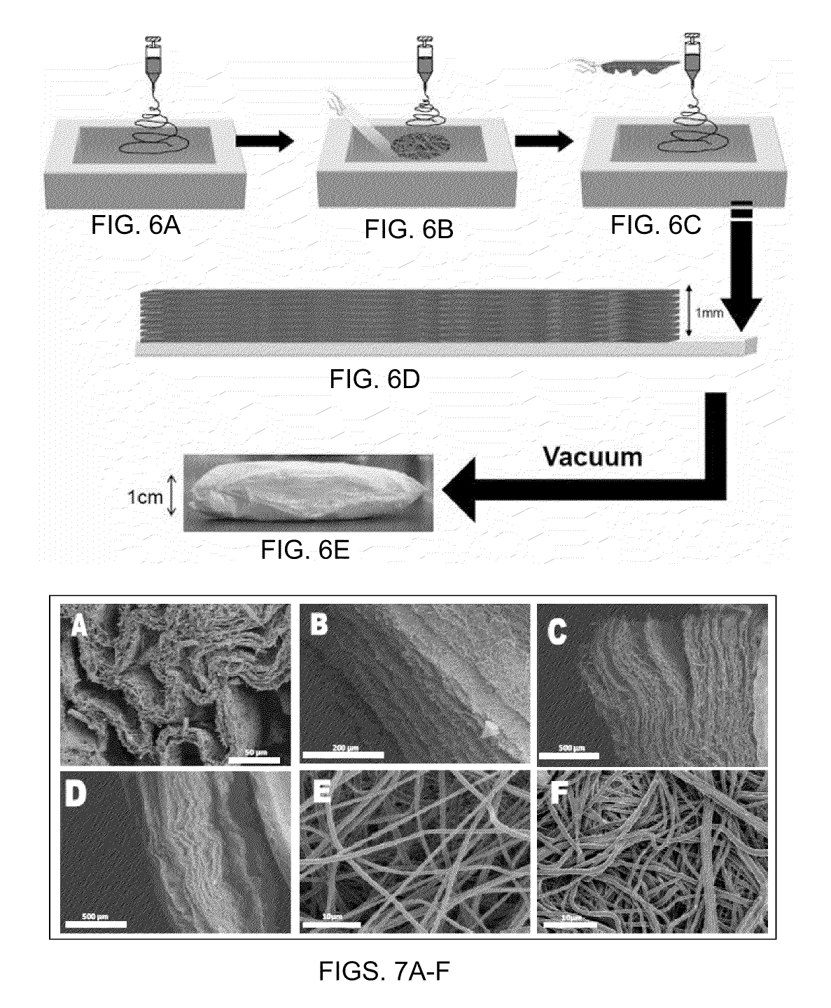

Referring now to the drawings, FIGS. 1A-C are schematic illustrations of a nonwoven structure 10, according to various exemplary embodiments of the present invention. Electron microscopy images of structure 10 according to some embodiments of the present invention are provided in the examples section that follows (see, e.g., FIGS. 6E, 7A-C and 8B).

Nonwoven structure 10 is preferably shaped as a sheet. It is to be understood that although structure 10 appears to be flat in FIG. 1A, this need not necessarily be the case since for some applications it may not be necessary for structure 10 to be flat. FIG. 1B schematically illustrates a perspective view of structure 10 in an embodiment in which structure 10 is non-planar.

Nonwoven structure 10 can be used in many applications, particularly, but not exclusively, clinical applications. For example, in some embodiments of the present invention structure 10 serves as or being a part of a scaffold for tissue engineering, e.g., tissue regeneration or tissue repair. Thus, structure 10 can be used for repairing bone defects caused by trauma, bone tumor resection, in the case of joint fusion and spinal fusion for non-healing fractures and osteoporotic lesions. Structure 10 can also be used in treating tooth and jaw defects in cases of trauma, bone loss, tooth loss, gum disease and the like. Structure 10 is also useful in treating cartilage defects such as those which result from rheumatoid arthritis, osteoarthritis and trauma. Structure 10 can also be used to repair defects and damage in skin, muscle and other soft tissues such as results from trauma, burns, ulcers (diabetic ulcers, pressure sores, venus, stasis ulcers, etc.). Structure 10 can also be utilized for treating damage to visceral organs including liver damage, heart attack damage, and damage resulting from intestinal cancer or intestinal ulcer.

Nonwoven structure 10 can also be used for supporting in vitro culture of living cells, for example, with the purpose of creating tissue constructs for repairing tissues and organs in vivo. Structure 10 may be used to promote tissue culture of committed cells and/or differentiation of precursor cells. Thus, structure 10 can be used in virtually all instances when it is desirable to provide a substrate for the growth of cells onto or into a tissue replaceable matrix. Structure 10 can also be used with autografts, allografts, and xenografts associated with bone grafts, cartilage grafts and joint resurfacing implants.

Nonwoven structure 10 is a multilayer structure which comprises a plurality of nonwoven layers 12 of fibers 14. Fibers 14 can be made of any material which can be fiberized, e.g., by a spinning technique and sustain a fibrous shape at a range of temperatures and pressures at which structure 10 is utilized. Typically, the range of temperatures is from about 0° C. to about 40° C. and the range of pressures is from about 100 Pa to about 100 kPa. A preferred method for fabricating a nonwoven structure, such as structure 10 is provided hereinunder.

Representative examples of materials suitable for the present embodiments include, without limitation, natural or synthetic polymers or co-polymers. The polymers can be biocompatible, biodegradable or biostable polymers.

The phrase “synthetic polymer” refers to a polymer that is not found in nature, even if the polymer is made from naturally occurring materials. Examples include, but are not limited to, aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, and combinations thereof.

Suitable synthetic polymers for use in the present invention can also include biosynthetic polymers based on sequences found in collagen, elastin, thrombin, fibronectin, starches, poly(amino acid), poly(propylene fumarate), gelatin, alginate, pectin, fibrin, oxidized cellulose, chitin, chitosan, tropoelastin, hyaluronic acid, polyethylene, polyethylene terephthalate, poly(tetrafluoroethylene), polycarbonate, polypropylene and poly(vinyl alcohol), ribonucleic acids, deoxyribonucleic acids, polypeptides, proteins, polysaccharides, polynucleotides and combinations thereof.

The phrase “natural polymer” refers to polymers that are naturally occurring. Non-limiting examples of such polymers include, silk, collagen-based materials, chitosan, hyaluronic acid and alginate.

The phrase “biocompatible polymer” refers to any polymer (synthetic or natural) which when in contact with cells, tissues or body fluid of an organism does not induce adverse effects such as immunological reactions and/or rejections and the like. It will be appreciated that a biocompatible polymer can also be a biodegradable polymer.

Non-limiting examples of biocompatible polymers include polyesters (PE), polycaprolactone (PCL), calcium sulfate, polylactic acid (PLA), polyglycolic acid (PGA), polyethyleneglycol (PEG), polyvinyl alcohol, polyvinyl pyrrolidone, polytetrafluoroethylene (PTFE, teflon), polypropylene (PP), polyvinylchloride (PVC), polymethylmethacrylate (PMMA), polyamides, segmented polyurethane, polycarbonate-urethane and thermoplastic polyether urethane, silicone-polyether-urethane, silicone-polycarbonate-urethane collagen, polyethyleneglycol-dimethylamine (PEG-DMA), alginate, hydroxyapatite and chitosan, blends and copolymers thereof.

The phrase “biodegradable polymer” refers to a synthetic or natural polymer which can be degraded (e.g., broken down) in the physiological environment such as by proteases. Biodegradability depends on the availability of degradation substrates (i.e., biological materials or portion thereof which are part of the polymer), the presence of biodegrading materials (e.g., microorganisms, enzymes, proteins) and the availability of oxygen (for aerobic organisms, microorganisms or portions thereof), carbon dioxide (for anaerobic organisms, microorganisms or portions thereof) and/or other nutrients. Examples of biodegradable polymers suitable for the present embodiments include, but are not limited to, collagen (e.g., collagen I or IV), fibrin, hyaluronic acid, PLA, PGA, PCL, polydioxanone (PDO), trimethylene carbonate (TMC), PEG, collagen, PEG-DMA, alginate, chitosan copolymers or mixtures thereof.

As used herein, the phrase “co-polymer” refers to a polymer of at least two chemically distinct monomers. Non-limiting examples of co-polymers suitable for the present embodiments include PLA-PEG, PEGT/PBT, PLA-PGA PEG-PCL and PCL-PLA.

According to an embodiment of the present invention, fibers 14 are made of more than one polymer. Each of these polymers can be a polymer or a co-polymer such as those described hereinabove. For example, the polymer of the present invention can comprise one or more biocompatible polymers and a co-polymer (either biodegradable or non-biodegradable).

In various exemplary embodiments of the invention fibers 14 comprise or are made of PCL.

Fibers 14 are preferably of micrometric or nanometric diameter. When fibers 14 are of micrometric diameter, the average diameter of the fibers is preferably from about 1 μm to about 20 μm, more preferably from about 5 μm to about 15 μm. When fibers 14 are of nanometric diameter, the average diameter of the fibers is preferably from about 100 nm to about 1000 nm, more preferably from about 500 nm to about 900 nm.

In some embodiments of the present invention, fibers 14 are of micrometric size and the thickness of a single layer of nonwoven structure 10 is from about 12 μm to about 18 μm. In some embodiments of the present invention, fibers 14 are of nanometric size and the thickness of a single layer of nonwoven structure 10 is from about 18 μm to about 12 μm.

Although in the above embodiments fibers 14 are at most microscopic in their diameter, structure 10 is substantially thick. In various exemplary embodiments of the invention the average thickness of nonwoven structure 10 is at least 0.5 mm, or at least 0.6 mm, or at least 0.7 mm, or at least 0.8 mm or at least 0.9 mm, e.g., 1 mm or more.

The average thickness of structure 10 can be determined by measuring the thickness of the structure, for example, by a caliper or the like, at several locations over the outermost layer of structure 10 and calculated the arithmetic average of the measurements.

High thickness for nonwoven structure 10 is useful when structure 10 serves as a scaffold for tissue engineering, particularly, but not exclusively, for repairing tissues such as bone having large defect.

In various exemplary embodiments of the invention structure 10 is substantially porous. Generally, the porosity of any solid structure can be defined as the percentage or fraction of the structure which is not occupied by solid matter. One way to determine the porosity is to calculate to the ratio between the density of the material from which the structure is made and the density of the structure itself, and to calculate the complementary value to this ratio. With such definition, the porosity P of the structure is given by the expression P=1−ρₘ/ρₛ, where ρₘ is the density of the material or composition from which the structure is made and ρₛ is the density of the structure itself. In various exemplary embodiments of the invention the overall porosity of nonwoven structure 10, when determined according to the above formalism and expressed in percentage, is at least 90%, or at least 91%, or at least 92%, or at least 93%, or at least 94%, or at least 95%, or at least 96%, or at least 97%, or at least 98% e.g., 99%. The high porosity of structure 10 is advantageous, for example, when structure 10 serves as a scaffold, since a highly porous structure allows cellular growth within the scaffold.

The high porosity of structure 10 can be achieved in more than one way. In some embodiments, high porosity is achieved by providing sufficiently large spaces between two or more of the individual layers forming structure 10, in some embodiments, high porosity is achieved by providing a structure with a sufficiently large number of layers, and in some embodiments, high porosity is achieved by providing a structure in which the individual layers are sufficiently porous. Each of these embodiments is further described hereinbelow separately, but it is to be understood that any combination of two or all of these embodiments is also contemplated.

In various exemplary embodiments of the invention at least two adjacent layers of structure 10 are at least partially spaced apart such at least two adjacent layers are distinguishable from one another (e.g., by means of electron microscopy, such as scanning or high resolution scanning electron microscopy, transmission or high resolution transmission electron microscopy, etc.). In various exemplary embodiments of the invention such two adjacent layers are distinguishable solely by the partial spacing therebetween. Specifically, in these embodiments, at least two adjacent layers of structure 10 have the same chemical and geometrical properties but are still distinguishable from one another since they are at least partially spaced apart. The chemical and geometrical properties of an individual layer of structure 10 refer to at least one of the following properties: type of fibers (material, average diameter), average density of fibers, and average pore size as delineated by fibers engaging the same layer.

Two adjacent layers can be spaced apart fully or partially. When the layers are fully spaced apart, there is a large void between the two layers, which void spans over the entire area of the layers, and serves for increasing the porosity. When the layers are partially spaced apart, there is still a void between the two layers, but the void spans over a portion of the layer's area. FIG. 1C is a schematic illustration of a cross-sectional view of structure 10 showing layers 12 and voids 16 between adjacent layers.

The voids between the various layers of structure 10 increase the average thickness per layer of structure 10. The average thickness per layer is defined as the average thickness as defined above, divided by the number of layers in structure 10. In some embodiments of the present invention the average thickness per layer is at least 10 times, or at least 20 times, or at least 30 times, or at least 40 times the average fiber diameter. In various exemplary embodiments of the invention the average thickness per layer is about 50 times the average fiber diameter.

In some embodiments of the present invention the overall thickness of structure 10 is at least K×N×T, where N is the number of layers in structure 10, T is the average thickness of a single layer, and K is a number which is at least 1.5 or at least 2 or at least 3 or at least 4.

Although structure 10 is shown in FIGS. 1A-C as having only a few layers (eight layers are shown in FIG. 1A, and five layers are shown in FIGS. 1B and 1C), this need not necessarily be the case. In various exemplary embodiments of the invention structure 10 comprises at least 50 layers, or at least 70 layers, or at least 90 layers, e.g., 100 layers or more. In various exemplary embodiments of the invention structure 10 comprises at least 160 layers, or at least 180 layers, or at least 200 layers, or at least 220 layers, e.g., 240 layers or more. As stated, a sufficiently large number of layers is advantageous since it facilitate high porosity of structure 10.

Another way for achieving high porosity of structure 10, is by providing it with a sufficiently porous individual layers. In these embodiments, at least a few of the layers of structure 10 have a sufficiently high average intra-layer pore diameter. Intra-layer pores of structure 10 are shown at 18. Unlike voids 16 which are defined between adjacent layers in structure 10, the intra-layer pores 18 engage the layers themselves and not the space between the layers. When fibers 14 are of micrometric size, the average intra-layer pore diameter is preferably at least 25 μm. When fibers 14 are of nanometric size, the average intra-layer pore diameter is preferably at least 4 μm, or at least 4.5 μm, or at least 5 μm.

Nonwoven structure 10 can serve as, or be a component of, a scaffold for tissue engineering. In various exemplary embodiments of the invention nonwoven structure 10 is a component in a cell-scaffold composition which comprises a scaffold and at least one cell population seeded on the scaffold. The scaffold can comprises structure 10 and the cell population(s) can be seeded on one or more of layers 12.

As used herein the term “cell population” refers to one or more cell populations.

In some embodiments of the present invention the cell population has a predetermined density distribution along a thickness direction z of structure 10. Any density distribution can be obtained. For example, in some embodiment the cell population has a generally uniform (e.g., within a standard deviation of 10% or less) density distribution along the thickness direction. In some embodiments the cell population has higher density at layers located generally the center of structure 10 and lower density at layers farther from the central layers. In some embodiments the cell population has higher density layers located near the outermost layers and lower density at layers located near the central layers. Also contemplated is a predetermined gradient of density distribution along the thickness direction.

In some embodiments of the present invention nonwoven structure 12 comprises having different type of cells at different depth of the structure. For example, the top layers of structure 12 can be seeded with cells that appear in the epidermis, and the bottom layers of structure 12 can be seeded with cells that appear in the dermis. Thus the cell-scaffold composition of the present embodiments can serve as an artificial skin tissue.

The cell population preferably occupies structure 10 at any depth within structure 10. It was found by the inventor of the present invention that structure 10 host the cell population throughout the thickness direction even when structure 10 is substantially thick, e.g., 1 cm or more.

Many types of cells are contemplated. Representative examples include, without limitation, a human embryonic stem cell, a fetal cardiomyocyte, a myofibroblast, a mesenchymal stem cell, an autotransplanted expanded cardiomyocyte, an adipocyte, a totipotent cell, a pluripotent cell, a blood stem cell, a myoblast, an adult stem cell, a bone marrow cell, a mesenchymal cell, an embryonic stem cell, a parenchymal cell, an epithelial cell, an endothelial cell, a mesothelial cell, a fibroblast, a myofibroblast, an osteoblast, a chondrocyte, an exogenous cell, an endogenous cell, a stem cell, a hematopoetic stem cell, a pluripotent stem cell, a bone marrow-derived progenitor cell, a progenitor cell, a myocardial cell, a skeletal cell, a fetal cell, an embryonic cell, an undifferentiated cell, a multi-potent progenitor cell, a unipotent progenitor cell, a monocyte, a cardiomyocyte, a cardiac myoblast, a skeletal myoblast, a macrophage, a capillary endothelial cell, a xenogenic cell, an allogenic cell, an adult stem cell, and a post-natal stem cell.

More specifically, when the structure of the present embodiments is used for repairing bone defects or tooth and jaw defects, it can be seeded with bone cells (osteoblasts and osteocytes) and/or bone cell precursors (mesenchymal stem cells from bone marrow, periosteum, endosteum, etc.); when the structure of the present embodiments is used for treating cartilage defects, it can be seeded with chondroblasts and chondrocytes and cartilage cell precursors such as the cell precursors described above in connection with bone; when the structure of the present embodiments is used for repairing defects and damage in skin, muscle and other soft tissues, it can be seeded with, for example, dermal fibroblasts, keratinocytes, and skeletal muscle cells; and when the structure of the present embodiments is used for treating damage to visceral organs it can be seeded with cells such as hepatocytes, cardiac muscle cells, intestinal cells, etc.

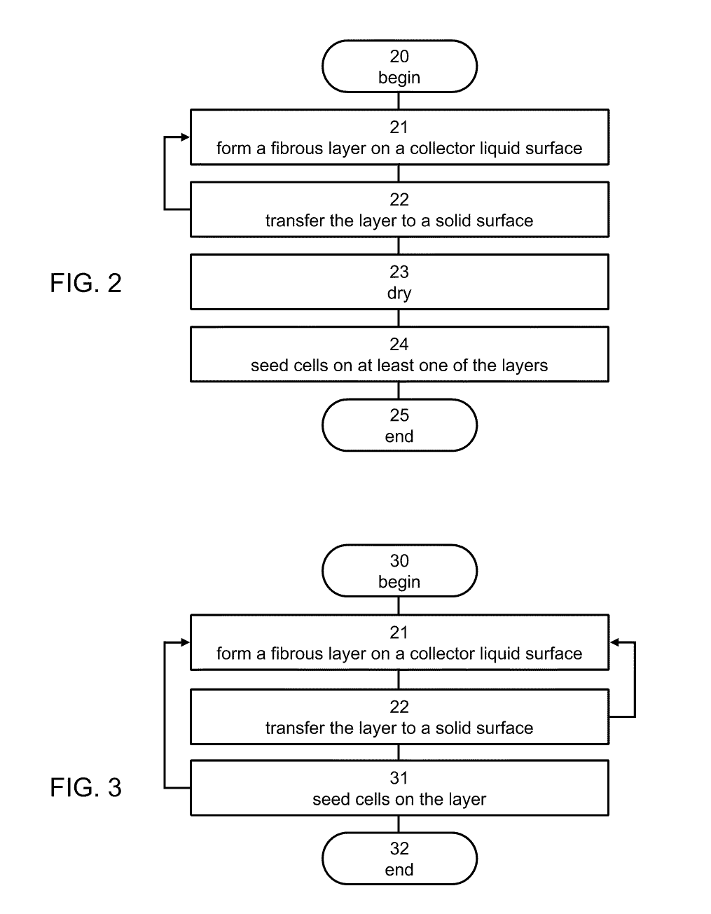

Reference is now made to FIGS. 2 and 3 which are flowchart diagrams of a method suitable for fabrication a nonwoven structure (e.g., structure 10), according to various exemplary embodiments of the present invention.

It is to be understood that, unless otherwise defined, the operation described hereinbelow can be executed in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

With reference to FIG. 2, the method begins at 20 and continues to 21 at which a nonwoven layer of polymer fibers is formed onto a surface of a collector liquid. The fibers are preferably of micrometric or nanometric diameter, as further detailed hereinabove.

As used herein, “liquefied polymer” refers to any polymer, polymers or co-polymers which are in a liquid form. For example, liquefied polymer can be a soluble polymer in solution or a melted polymer. The liquefied polymer can include any of the polymers described above with respect to structure 10. Other polymers or co-polymers are not excluded from the scope of the present invention.

In various exemplary embodiments of the invention the layer is formed by spinning a liquefied polymer onto a surface of a collector liquid. The spinning can be effected by electrostatic forces, optionally in combination with hydrostatic forces. The collector liquid serves as a coagulation medium for the spun fibers. The advantage of using a coagulation medium is that it initiates a formation of skin surrounding the fibers, and thus prevents or reduces fiber fusion.

Typically, the collector liquid surface is at room temperature (about 20° C.), but this need not necessarily be the case, since, for some applications, it may be desired to maintain the collector liquid at other temperatures.

Many types of collector liquid are contemplated. In some embodiments of the present invention the collector liquid is an aqueous liquid, such as, but not limited to, distilled water.

In some embodiments of the present invention the collector liquid is an organic solvent, such as DMSO, methanol, ethanol, THF, DMF, propylene glycol, etc. In these embodiments, the liquefied polymer is preferably in a hydrophilic solution.

In some embodiments of the present invention the collector liquid comprises saline. For example, the collector liquid can be a culture medium. These embodiments are particularly useful when the structure is fabricated to serve as a scaffold for supporting cell growth, e.g., a scaffold for tissue engineering. Any type of culture medium can be used, including, without limitation, a medium which comprises at least one of: salts, sugars, amino acids and minerals in the appropriate concentrations and with various additives. Those of skills in the art of cell growth will know how to determine a suitable culture medium for specific cell types. A non-limiting example of a culture medium suitable for the present embodiments include, phosphate buffered saline (PBS). PBS is a standard biological solvent composed of 0.9% NaCl in water, adjusted to a pH 7.2 with phosphate buffer. Other examples for culture media include, without limitation, MEM-α and DMEM (Dubelco's Modified Eagle Medium). The culture medium can also be supplemented with various antibiotics (e.g., penicillin and streptomycin), growth factors or hormones, cytokines and the like.

Spinning techniques suitable for the present embodiments are found in the literature, see, for example, U.S. Pat. No. 7,354,546; Srinivasan G and Reneker D., “Structure and morphology of small diameter electrospun aramid fibers,” Polymer International 1995, 36(2): 195-201; Kim et al., “Structural studies of electrospun cellulose nanofibers,” Polymer 2006, 47(14):5097-5107; Smit et al., “Continuous yarns from electrospun fibers,” Polymer 2005, 46(8):2419-2423; and Teo et al., “A dynamic liquid support system for continuous electrospun yarn fabrication,” Polymer 2007, 48(12):3400-3405, the contents of each of which are hereby incorporated by reference.

The process in which fibers are spun onto a collector liquid surface is referred to herein as “hydrospinning.” A hydrospinning process according to some embodiments of the present invention is described hereinunder and further exemplifies in the Examples section that follows.

The method continues to 22 at which the layer is transferred to a solid surface, such as a glass plate or the like, and loops back to 21 to form the structure in a layerwise manner. The formation of the structure is “layerwise” in the sense that each newly formed layer is piled onto a previously formed layer (except, of course, the first layer which is placed directly on the solid surface). During the transfer of the newly formed layer from the surface of the liquid to the solid surface, the spinning is preferably temporarily terminated. Thus, the present embodiments apply an intermittent and periodic spinning process.

It was found by the inventors of the present invention that the above layerwise process can facilitate high formation rates for the structure. Generally, the formation rate of the multilayer nonwoven structure of the present embodiments depends on the spinning parameters (accelerating forces, flow rate of liquefied polymer, geometry of the spinneret device, and the like) as well as on the rate at which a newly formed layer is collected off the liquid surface. In various exemplary embodiments of the invention the spinning parameters and the collection rate of the layers off the surface are selected such that the multilayer structure is formed at a rate of at least 20 mm³/min, more preferably at least 40 mm³/min, more preferably at least 60 mm³/min more preferably at least 80 mm³/min. It was found by the inventors of the present invention that these rates are achievable even for a spinning rate of less than 30 ml or less than 20 ml or less than 10 ml or less than 5 ml of liquefied polymer per hour.

Prior to, during or subsequently to the spinning process the method optionally and preferably incorporates one or more agents (e.g., chemical agents, biological agents, mineral agents and the like) in the multilayer structure. The agents can be incorporated by any technique known in the art. For example, the agents can be sprayed on one or more of the layers after their transfer to the solid surface. The agents can also be mixed with the liquefied polymer prior to the spinning process, thereby effecting embedding of the agents in the fibers. The agents can also be in the form of particles, which may externally attached to the fibers, for example, by spraying them onto one or more of the layers after their transfer to the solid surface. The particles may also be mixed with the liquefied polymer prior to the spinning process, thereby effecting formation of fibers impregnated with the particles. The agents (in fluid or particulated form) can also be mixed with the collector liquid such that once the fibers are collected by the liquid surface the agents are attached to the fibers.

In some embodiments of the present invention the agent or agents are selected for promoting proliferation of one or more population of living cells. This embodiment is useful when the structure is fabricated to serve as a scaffold for supporting cell growth, e.g., a scaffold for tissue engineering. Any type of culture medium agent can be used, including, without limitation, salts, sugars, amino acids and minerals in the appropriate concentrations and with various additives. Those of skills in the art of cell growth will know how to determine a suitable agent for specific cell types. The agents can also include an antibiotic, a growth factor or a hormone, a cytokine and the like.

Optionally and preferably the method continues to 23 at which the multilayer structure is dried. This can be done, for example, by a vacuum desiccating procedure whereby the multilayer structure in subjected to vacuum conditions for a time period which is sufficient to desiccate the structure. Typical vacuum conditions suitable for the present embodiments are pressure of less than 100 mbar for a period of a few hours to a few days. In experiments performed by the inventors of the present invention, a pressure of 20 mbar and a time-period of 4 days were selected.

The incorporation of agents in the multilayer structure can, In some embodiments of the present invention, be executed subsequently to the drying operation (in embodiments in which this operation is executed). For example, subsequently to the drying operation, the multilayer can be placed in a culture medium for promoting proliferation of one or more population of cells.

The method of the present embodiments can also include a stage in which the multilayer structure is sterilized. This is preferably done subsequently to the spinning process and, in operation in which the structure is dried, subsequently to the drying operation. For example, the structure can be immersed in a sterilization solution (e.g., ethanol or the like). Those skilled in the art would know how to select a sterilization solution or sterilization technique which is appropriate for the application for which the structure is fabricated.

In some embodiments of the present invention the method proceeds to 24 at which living cells are seeded on at least one of the layers of the structure. The seeding can be applied to one of the outermost layers of the structure, or it can be applied directly to one or more of the internal layers of the structure. A suitable method for the latter embodiment is described hereinafter with reference to FIG. 3.

The type or types of cells which are seeded at seeding operation 24, depends on the application for which the multilayer structure is used. To this end, any type of cell can be seeded, including, without limitation, any of the cells described above with respect to nonwoven structure 10.

The method ends at 25.

FIG. 3 is a flowchart diagram describing a method suitable for fabrication a nonwoven structure (e.g., structure 10), in embodiment of the invention in which cells are seeded on an internal layer of the structure.

The method begins at 30 and continues to 21 at which a layer is formed on a surface of a collector liquid, and 22 at which the layer is transferred to a solid surface, as further detailed hereinabove. In some embodiments of the present invention the method proceeds to 31 at which cells are seeded on the newly formed layer. From 31, the method, in various exemplary embodiments of the invention, loops back to 21 such that the structure is formed in a layerwise manner, as further detailed hereinabove. The seeding operation 31 is preferably performed for at least one internal layer of the structure. More preferably, two or more, preferably all the layers are seeded with the cells. Yet, some layers may not be seeded. For such layers, the method loops back from 22 to 21 without executing 31. Thus the present embodiments provide a layerwise seeding technique in which a newly formed and seeded layer is piled onto a previously formed and optionally seeded layer.

The layerwise seeding technique of the present embodiments allows the generation of a predetermined density distribution of cells along the thickness direction of multilayer structure. This is because the present embodiments allow control on the density of cells on each of the layers, thus facilitate patterned seeding. The density distribution of cells along the thickness direction can be any of the density distributions described above with respect to structure 10.

The layerwise seeding technique of the present embodiments also allows the fabrication of multilayer structure having different type of cells in different depth of the structure. For example, cells that appear in the epidermis can be seeded onto the top layers and cells that appear in the dermis can be seeded onto the bottom layers, thus fabricating an artificial skin tissue.

The method ends at 32.

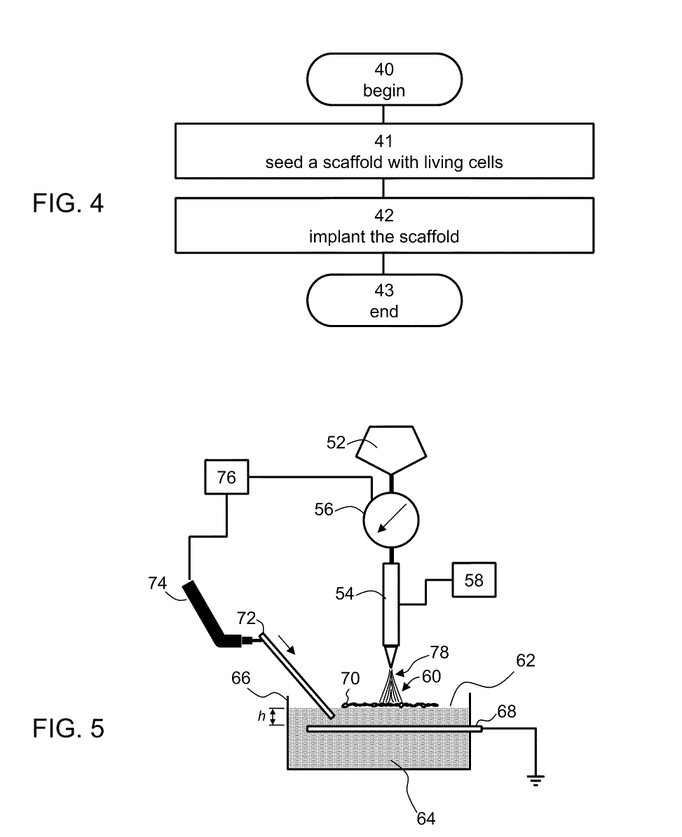

Reference is now made to FIG. 4 which is a flowchart diagram describing a method suitable for treating a patient, according to various exemplary embodiments of the present invention.

A “patient”, as used herein, refers to any life form, particularly mammals, including, human beings as well as animals, such as, but not limited to, dogs, cats, horses, cattle, sheep and the like are patients within the context of this invention.

The method begins at 40 and optionally and preferably continues to 41 at which a scaffold is seeded with cells as further detailed hereinabove so as to provide a cell-scaffold composition. In various exemplary embodiments of the invention the scaffold comprises structure 10. The method continues to 42 at which the cell-scaffold composition is implanted in the patient. The cell-scaffold composition is implanted at the site of the patient at which there is a repairable tissue defect. Representative examples of such sites include, without limitation, a defected bone, a defected tooth, a defected jaw, a defected cartilage (e.g., a cartilage in a damaged joint such as a knee, hip, or shoulder), a damaged skin, a damaged muscle and a damaged visceral organ (e.g., liver, heart, intestinal).

It is anticipated that a surgeon who is ready to commence the implanting operation of the present embodiments can be provided with a ready for use cell-scaffold composition contained in a sealed sterile package. In these situations, seeding operation 41 is not executed by the surgeon.

It is further anticipated that the surgeon can selects from an assortment of scaffolds, with a variety of different sizes, shapes, each of which is contained in a sealed sterile package. Once the surgeon has accessed the site of implantation, and has inspected the defect to see exactly how large it is, the surgeon can select a scaffold from the assortment that is available, and select a scaffold having the best size and shape for treating that particular defect.

The surgeon can then prepare the area by removing a relatively small amount of existing tissue which surrounds the defect, to prepare an exposed surface that can support the cell-scaffold composition. Once the surface has been properly prepared, the cell-scaffold composition can be positioned in the prepared area. All these operations are well known to those skilled in the art of clinical implantation.

The method ends at 43.

Reference is now made to FIG. 5 which is a schematic illustration of a system 50 for spinning polymer fibers on a collector liquid surface, according to various exemplary embodiments of the present invention.

System 50 can comprise a main container 52 for holding therein a liquefied polymer. The liquefied polymer is fed from container 52 to one or more spinnerets 54 (only one is shown for clarity of presentation). In various exemplary embodiments of the invention liquefied polymer is fed in an intermittent fashion so as to facilitate the aforementioned layerwise formation of nonwoven structure. Spinneret 54 can be provided as a nozzle or a needle. In experiments performed by the present inventor a 21 and 25 gauge needles were employed, but other diameters are also suitable. Optionally, system 50 comprises a metering pump 56 through which the liquefied polymer is injected into spinneret 54. Spinneret 54 is preferably electrically connected to a high voltage generator 58, typically to the positive terminal thereof. Typically, generator 58 generates a voltage of at least 5 kV. In experiments performed by the present inventors, voltages of 18.06 kV and 13 kV were employed, but other voltages are also suitable.

System 50 further comprises a collector container 66 holding a collector liquid 64 which can be an aqueous liquid, an organic solvent, a culture medium, etc., as further detailed hereinabove.

Collector container 66 preferably has a construction whereby a conductive material 68 which is preferably submerged collector liquid 64. Conductive material 68 is preferably grounded with respect to generator 58 such that there is a high potential difference between spinneret 54 and collector container 66. Other electrostatic configurations (such as a configuration in which spinneret 54 is grounded and conductive material 68 is kept at a high voltage of negative polarity relative to spinneret 54) are also contemplated. Conductive material 68 can be metal plate or metal powder. Also contemplated are configuration in which collector container 66 is made of a conductive material. In these embodiments, collector container 66 can be grounded or electrically connected to generator 58, to generate electric field between spinneret 54 and collector container 66.

In use, generator 58 is activated and an electric field is generated between spinneret 54 and collector container 66. The liquid polymer is injected into spinneret 54 and extruded from its aperture. Charged jets 78 depart from the spinneret 54 and travel within the electric field towards collector container 66. The solvent in jet 78 evaporates resulting in formation of spun fibers 60 moving in the direction of surface 62 of collector liquid 64. Fibers 60 contact the liquid collector and a layer 70 of spun fibers 60 is formed on surface 62.

The distance from the surface 62 of liquid 64 to the top surface of conductive material 68 is preferably selected sufficiently large such as to avoid contact between fibers 60 and conductive material 68, yet sufficiently small so as to ensure efficient collection of fibers 60 on surface 62. Typical distance from the surface 62 to the top surface of conductive material 68 is, without limitation, about 5 cm.

The distance from the surface 62 to spinneret 54 is preferably selected sufficiently large to allow evaporation of at least a portion of the solvent in jet 78 while jet 78 moves from spinneret 54 to surface 62, yet sufficiently small so as to establish an electric field which is sufficient strong for pulling jet 78 out of spinneret 54. Typical distance from the surface 62 of liquid 64 to spinneret 54 is, without limitation, from about 5 cm to about 30 cm.

The flow rate of the liquefied polymer in spinneret 54 is preferably selected sufficiently high to allow extrusion of the liquefied polymer out of the aperture of spinneret 54, yet sufficiently low so as not to generate too thick fibers. Typical flow rates of the liquefied polymer in spinneret 54 are, without limitation, from about 0.5 ml/hr to about 20 ml/hr.

One of ordinary skill in the art of spinning will know how to adjust the above parameter for the desired properties of layer 70.

System 50 may also comprise a solid surface 72, such as a glass or plastic surface, for collecting layer 70 off liquid surface 62 and transferring to solid surface 72. The transfer of layer 70 can be done manually by submerging solid surface 72 into liquid 64 and lifting it up together with layer 70, once layer 70 is formed. The transfer of layer 70 can also be facilitated with a robotic arm 74 which can be configured to hold solid surface 72, submerging it into and move generic it liquid 64 and lifting it up together with layer 70. System 50 can also have a control unit 76 which can be configured to synchronize between the spinning and layer transfer operations. For example, unit 76 can be configured to ensure an intermittent spinning process. Specifically, unit 76 can send signals to generator 58, pump 56 and/or spinneret 54 to temporarily cease the spinning process while at the same send signals to robotic arm 74 to transfer the newly formed layer to solid surface 72. Once robotic arm 74 completes the transfer, unit 76 can send signals to generator 58, pump 56 and/or spinneret 54 to continue the spinning process.

As used herein the term “about” refers to ±10%.

The word “exemplary” is used herein to mean “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

### Fabrication of Prototype Scaffolds

Two types of prototype multilayered scaffolds were fabricated via intermittent hydrospinning in accordance with some embodiments of the present invention. These scaffolds are referred to hereinbelow as type I and type II. The scaffolds were analyzed and compared to control scaffolds prepared from the same material and in similar electrostatic configuration but via continuous electrospinning.

### Material and Methods

### Hydrospun Scaffolds

In type I scaffold, the materials for the hydrospinning included a 15% solution of poly(caprolactone) (PCL) (M_{W} 80,000, Aldrich) prepared in a 75:25 mixture of dichloromethane (DCM) (Frutarom) and Dimethilformamide (DMF) (Frutarom). Hydrospinning voltage of 18.06 kV was generated by DC Power supply (Glassman High Voltage LTD). A 21 gauge needle (stubs needle gauge) (BD Microlance™ 3, LOT 0607) was used as a spinneret, and the collector liquid surface was distilled water at room temperature with a 1.25 gr/L concentration of NaCl. The distance between the spinneret and the collector liquid surface was 32.5 cm, and the flow rate was 20 ml/hr. A layer of spun fibers was collected off the liquid surface every 10 seconds.

In type II scaffold, the materials for the hydrospinning included a 10% solution of PCL prepared in a 75:25 mixture of DCM and DMF. Hydrospinning voltage of 13 kV was generated by DC Power supply. A 25 gauge needle was used as a spinneret. The distance between the spinneret and the collector liquid surface was 20 cm, and the flow rate was 1.2 ml/hr. A layer of spun fibers was collected off the liquid surface every 60 seconds. The collector liquid surface and the vendors of the materials and equipment were the same as in type I.

Both the above types of scaffolds were desiccated in a vacuum environment (20 mbar) for 2 days.

The hydrospinning process employed in the present example is illustrated in FIGS. 6A-E. FIG. 6A illustrates the spinning onto a collector liquid surface (water in the present example), FIG. 6B illustrates the formation of a thin layer of spun fibers on the surface of the liquid and the collection of the layer on a solid surface (glass surface in the present example) and off the liquid surface, FIG. 6C illustrates the formation of a new layer on the collector liquid surface. The operation is repeated and each layer is collected on top of a previously collected layer to thereby assemble a multilayer scaffold as illustrated in FIG. 6D. Once the multilayered scaffold is dried (vacuum desiccated in the present example), its size is increased, as shown in FIG. 6E. In the exemplified embodiment shown in FIGS. 6D and 6E, the multilayer scaffold multiplies ten times in thickness (from about 1 mm to about 1 cm) during vacuum desiccation.

### Control Scaffolds

For comparison, two types of control scaffolds were fabricated using the same parameters as above with respect to the type I and type II scaffolds, but via a continuous electrospinning process instead of intermittent hydrospinning process. The control scaffolds are referred to below as type I control scaffolds and type II control scaffolds, respectively.

The collector in the electrospinning process was a flat electrode covered with aluminum-foil.

Similarly to the type I and type II scaffolds, the control scaffolds were desiccated in a vacuum environment (20 mbar) for 2 days.

### Scaffold Analysis

Following desiccation, the scaffolds (two types of hydrospun scaffolds and two types of control scaffolds) were sputter-coated with gold (Polaron Equipment LTD, E5150, SEM Coating Unit) and observed by scanning electron microscopy (HR-SEM, LEO 982) at an accelerating voltage of 3 or 4 kV. The HR-SEM images were analyzed using SemAfore (SemAfore Version 4.01), and the fibers diameter and pore diameter was recorded. To minimize bias when choosing which pores to measure, only points along a line drawn from the upper-right to the lower-left corners of the each image were counted.

The porosity of the scaffold was determined via gravitometric measurements. The geometry of each scaffold was measured using a caliper. The porosity P of the each scaffold was determined by weighing the scaffold, calculating its density ρ, and using the formula

P=1−ρ_{scaffold}/ρ_{PCL},

where ρ_{PCL} is the density of the PCL.

In order to quantify and standardize the fabricated scaffold's volume in relation to the spinning time, a Scaffold Buildup Rate (SBR) was defined as follows:

SBR=l×w×v

where l and w are the length and width of the scaffold, respectively, and v is the rate at which the scaffold's thickness grows after vacuum treatment.

### Results

The average diameter of the fibers and the average diameter of the pores in the fabricated scaffolds is summarized in Table 1, below, along with the measured porosity and thickness of each scaffold.

TABLE 1

| | average | average | | | average | | |
|---|---|---|---|---|---|---|---|
| | fiber | pore | average | | scaffold | spinning | SBR |
| | diameter | diameter* | porosity | number | thickness | duration | [mm³/ |
| | [μm] | [μm] | [%] | of layers | [mm] | [min] | min] |
| type I control | 6 ± 1.4 | 20.7 ± 9.3 | 68.4 | — | 0.202 | 45 | 8.9 |
| type I | 8.3 ± 1.2 | 26.6 ± 10.9 | 93.4 | 100 | 0.74 | 16.6 | 88 |
| type II control | 0.6 ± 0.14 | 2.8 ± 1.7 | 80.4 | — | 0.29 | 690 | 0.8 |
| type II | 0.8 ± 0.19 | 5.3 ± 3.1 | 99.3 | 240 | 10 | 240 | 82.3 |

*for type I and type II scaffolds, the data in this column refer to the average diameters of intra-layer pores.

The data in Table 1 indicate that the average pore size is scaffolds prepared according to exemplary embodiments of the present invention. The data in Table 1 also show that the average thickness of scaffolds prepared according to exemplary embodiments of the present invention is substantially higher than the average thickness of the control scaffold. For the particular scaffolds presented in this example, the thickness of the type I scaffold is more than 3 times higher than the thickness of the type I control scaffold, and the thickness of the type II scaffold is more than 34 times higher than the thickness of the type II control scaffold.

Additionally, the scaffold buildup rate, SBR, is substantially higher for the scaffolds of the present embodiments than for the control scaffolds. For the particular scaffolds presented in this example, the SBR of the type I scaffold is about an order of magnitude higher than the SBR of the type I control scaffold, and the thickness of the type II scaffold is about two orders of magnitude higher than the SBR of the type II control scaffold.

The scaffolds of the present embodiments are formed of a plurality of identifiable layers (in the present example, 100 layers for the type I scaffold 240 layers for the type II scaffold).

FIGS. 7A-D are SEM images showing cross-sections of the multilayer scaffolds of the present embodiments (FIGS. 7A-C) and the control scaffolds (FIG. 7D). FIG. 7A shows a cross-section of type II scaffold, FIGS. 7B and 7C show cross-sections type I scaffold, and FIG. 7D shows a cross-section of type I control scaffold. As shown, large voids are formed between adjacent layers of the scaffold of the present embodiments. FIGS. 7E and 7F are SEM images of hydrospun fibers of nanometric size and electrospun fibers of nanometric size, respectively.

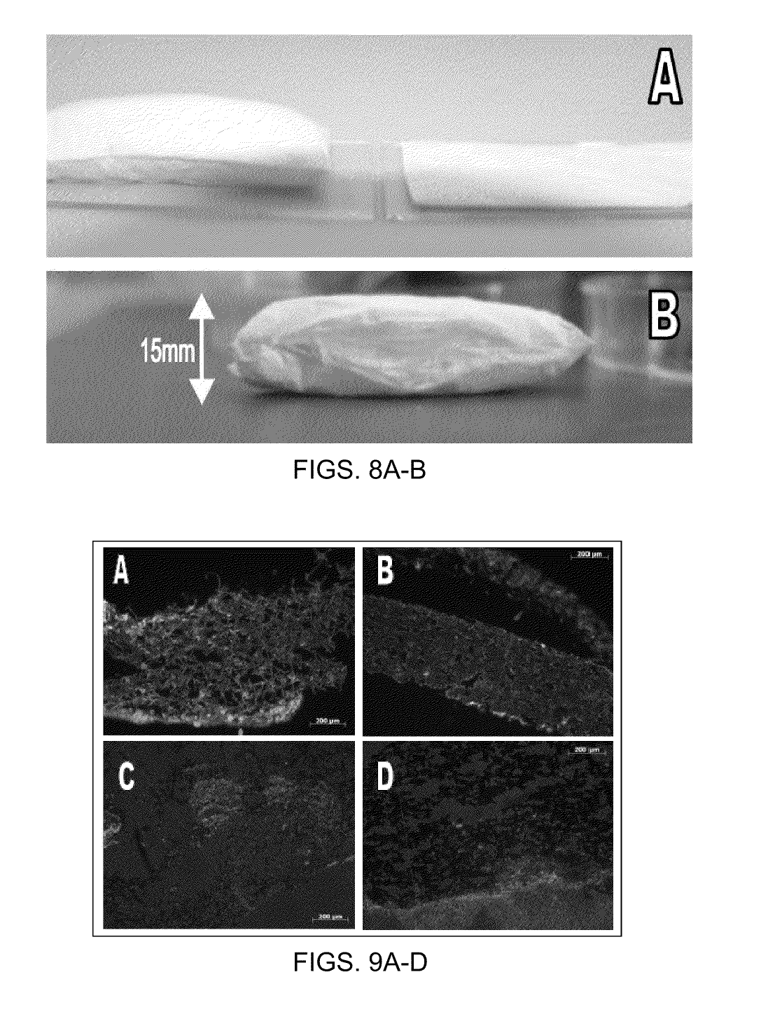

FIG. 8A shows SEM images of the type II scaffold of the present embodiments (left) and the type II control scaffold (right). The difference in thickness between the layers is evident. FIG. 8B shows the type I scaffold of FIG. 8A, cut open to reveal its multilayer structure.

Without being bound to any theory, it is postulated that when the layers of the scaffolds of the present embodiments are piled one over the other, large voids are formed between wrinkles of adjacent layers. During the scaffold buildup, the voids are filled with the liquid on which the layers are hydrospun. When the liquid is dried (e.g., vacuum desiccated, as in the present example), the entrapped liquid is evacuated from the scaffolds through the layers, and drag forces are generated between the liquid and the layers. As a result, the layers are stretched and extended in all three dimensions, including the thickness dimension. Even when the scaffolds of the present embodiments are removed from the vacuum environment, the voids between adjacent layers are retained (see, for example, FIG. 8B).

The above description is consistent with the differences in overall thickness between the scaffolds of the present embodiments and the control scaffolds. Thus, a scaffold which is made of a plurality of layers is generally thicker than an equivalent scaffold (e.g., made the same material, formed of fibers of generally the same diameter and having generally the same overall weight) made of a single layer.

The above description is also consistent with the differences in overall thickness between the type I and type II scaffolds of the present embodiments. The fibers forming the layers of the type I scaffold are thicker than the fibers that form the layers of the type II scaffold (about 10 times thicker, in the present example). Thicker fibers are more permeable to the liquid, and the drag forces between the liquid and the layers during liquid evacuation are weaker.

Thus, a multilayer scaffold made of thinner fibers is generally thicker than an equivalent multilayer scaffold (e.g., made the same material and having generally the same overall weight) made of made of thicker fibers.

The voids between the stretched layers of the scaffolds of the present embodiments are actually extremely large pores that add to the overall porosity of the scaffold and can enable cellular growth.

Another difference between the scaffolds of the present embodiments and the control scaffolds is in the average diameter of intra-layer pores (namely, pores that are formed within the layer, which are not to be confused with the aforementioned voids between layers). Without being bound to any theory, it is postulated that the average intra-layer pore diameter of the control scaffold is smaller due to the fact that the fibers are still electrically charged when they fall onto the conductive collector. Since the collector is solid, the fibers are gathered one on top of the other, and retain sufficient residual charges to repel each other. The (charged) fibers on the boundary of the pore repel the next collected fiber approximately to the middle of the pore, where the vector sum of all repulsion force is minimal. Schematically, each new fiber cuts a pore size down by half. In the hydrospun scaffolds of the present embodiments, on the other hand, the fibers are formed on a liquid medium, and are therefore immediately discharged. The created intra-layer pores of the hydrospun scaffold of the present embodiments are thus larger than those of the control scaffolds.

### Example 2

### Fabrication of Prototype Seeded Scaffolds

Cells were seeded onto scaffolds prepared substantially as described in Example 1 above. Two different types of cells were seeded, and two different seeding techniques were employed. The seeded cells were allowed to grow within the scaffolds. Subsequently, the scaffolds were sectioned, stained and analyzed.

### Material and Methods

### Layerwise Seeding

A type I scaffold with 100 layers was fabricated as described in Example 1 above, but using a phosphate buffered saline (PBS) with 1% antibiotic instead of distilled water as a collector liquid surface.

During fabrication of the scaffold, mouse myoblasts were seeded upon each layer, before collecting the next layer thereon. About 10,000 cells were seeded in a specific location on each layer. Once 100 layers were obtained, the scaffold was cut and put into a 6-wells plate, and a minimal amount of growth medium was added to prevent cellular death. After 30 minutes, 5 ml of growth medium were added to the well. The scaffold was incubated for 4 days, and the medium was replaced every day.

### Post-Spinning Seeding

hES cells (H9 clone) were grown on mouse embryonic fibroblasts (MEF) in knockout medium composed of 15% Knockout SR (Knockout SR, Gibco, 314796), 1% non-essential amino acids (MEM NEAA, Gibco, 301597), 100 mM L-Glutamine (L-Glutamin 200 mM, Gibco, 9825), 0.1M β-mercaptoethanol (2-mercaptoethanol 50 MM, Gibco, 208665), 4 ng/ml bFGF (Invitrogen, 13256-029) and 85% knockout D-MEM (Knockout DMEM, Gibco, 307381). Tissue culture plates were coated with 0.1% gelatin (Sigma). Cultures were grown in 5% CO₂ and were routinely passaged every 5-6 days after disaggregating with 1 mg/ml collagenase type IV (GIBCO/BRL). To induce formation of EBs, hES colonies were digested using 1 mg/ml collagenase type IV and transferred to Petri dishes to allow their aggregation and prevent adherence to the plate. Human EBs were grown in the same culture medium without basic fibroblast growth factor (bFGF).

Mouse skeletal myoblast cells (C₂), provided by Prof. David Yaffe of the Weitzman Institute, Israel, were maintained in culture medium, composed of 20% FBS (Fetal bovine serum, Hyclone, CPL0252), 1% penstrep (Pen-strep solution, Biological Industries, 652564), 2.5% HEPES (Hepes buffered saline solution, Clonetics, 01114089) and 76.5% D-MEM (DMEM, Gibco, 253810). The cells were harvested using 2 ml Trypsin-EDTA (0.5% Trypsin-EDTA, Gibco), and were not allowed to grow past 80% confluence.

Unseeded type I, type II, type I control and type II control scaffolds were fabricated as described in Example 1 above. Circular scaffolds were cut out of scaffolds using a puncher. The radius of the circular scaffolds was 0.4 cm. The type II scaffold and type II control scaffold were sterilized by an immersion overnight in a 70% ethanol (Ethanol absolute, Bio Lab LTD, 5565 11) solution. The type I scaffolds and type I control scaffolds were sterilized by plasma in a DEKAM device, at 200 RF for 30 seconds. The scaffolds were checked every two days throughout the experiment, to make sure they were not contaminated.

Following sterilization, the scaffolds were immersed for one hour in a solution of fibronectin 0.02 mg/ml (Fibronectin, from human plasma, Sigma, 066K7555). The scaffold were then washed twice with medium and seeded with 10⁶ cells on the upper surface each scaffold in culture wells.

An alternative method involved mixing the cells with a mixture of 1:1 medium and growth-factor reduced matrigel prior to the seeding them.

In either method, the seeded cells were given 30 minutes to adhere to the scaffolds, before a 5 ml medium was added to each well. The 6-wells plates were placed on a shaker and incubated in a temperature of 37° C., and 5% CO₂ in the air. The medium was replaced every two days.

### Post-Seeding Procedure

After 14 days of growth the seeded scaffolds were removed from the medium and washed twice with PBS. Thereafter, the scaffold were frozen in liquid nitrogen and cryo-sectioned with OCT (Tissue-Tek, O.C.T Compound, Sakura, 45 83).

The sectioned scaffolds were mounted on slides, and fixed with paraformaldehyde 3.2% (Diluted in PBS) for 30 minutes, followed by triton X-100 1% (Diluted in PBS) and a blocking serum of 10% FBS. The sectioned were then incubated for 30 minutes with a primary antibody, followed by a 30 minutes incubation with a secondary antibody, and a 10 minutes incubation with DAPI (Diluted in PBS to a 2.5 μg/μl concentration; 4,6-diamidino-2-phenylindole dihydrochloride, Sigma-Aldrich, D9542), or a 30 minutes incubation with FITC-conjugated phalloidin (Diluted in PBS to a 0.02 μg/μl-0.016 M, Sigma-Aldrich, P5282).

The primary antibodies used for the staining were mouse anti-desmin (clone D33, Dako Cytomation) and mouse anti-vimentin (clone V9, Dako Cytomation) and the secondary antibody was CY3 (F(ab)₂ goat anti-mouse igG-Cy3, Jackson Immunoresearch Laboratories Inc, 115-166-072). Alternatively, for histological examination, slides were stained with safranin-O or hematoxilin and eosin.

For SEM observation, the scaffolds were washed twice with PBS, incubated for 5 minutes in 2.5% glutaraldehyde in 0.1 M cocadylate buffer (Glutaraldehyde, Merck, S4226403 446) and then dehydrated with an upgrading series of ethanol. They were then incubated for 15 minutes in HDMS (Hexamethyldisilazane, Sigma, 044K0613), and left overnight to dry. The scaffolds were cross-sectioned, mounted on stubs and sputter coated with gold (Polaron Equipment LTD, E5150, SEM Coating Unit). Cell morphologies on the scaffolds were observed by a high-resolution scanning electron microscope (HR-SEM, LEO 982) at an accelerating voltage of 3 or 4 kV.

Images of the sectioned scaffolds were taken using a fluorescent microscope (Zeiss). A count of the cell nuclei appearing inside the scaffold was performed using the auto-count feature, tracking the DAPI stained cells. The count was performed only on the cells that penetrated into the scaffold itself, and not the cells covering the scaffold. 2-4 sections of each scaffold were examined, and the count was performed on two different areas of each section of the scaffold: the first 200 μm to either side, and the area at the middle of the section, at a depth of more than 200 μm. The number of cell nuclei in those areas was counted, and the average density of cells was calculated in each area.

### Results

### Cellular Growth Following Post-Spinning Seeding

The results of post-spinning seeding technique as employed for type I scaffolds and type I control scaffolds are shown in FIGS. 9A-D.

FIGS. 9A and 9B show a type I scaffold of the present embodiments (FIG. 9A) and a type I control scaffold (FIG. 9B), seeded with myoblasts and stained with anti-desmin (red), phalloidin (green) and DAPl (blue).

The cells in the scaffold of the present embodiments exhibited better and healthier staining for desmin. Moreover, due to the thickness of the scaffold of the present embodiments (in this example, more than two times thicker than the control scaffold), many more cells can inhabit the scaffold (see FIG. 9A).

The myoblasts were able to infiltrate deeply into the scaffolds and proliferate in them.

Following myoblast growth, the average thickness of the type I control scaffold was about 400 μm, and the density of infiltrated cells was calculated in depth of 200 μm from each side of the scaffolds. On the other hand, the average thickness of the type I scaffold of the present embodiments was about 1000 μm (following myoblast growth). For these scaffolds, the density of infiltrated cells was calculated both in depth of 200 μm from each side of the scaffold, and in the middle of the scaffold.

The cellular density in 200 μm depth was 8.07×10⁻⁴ myoblasts per square micrometer for the type I control scaffolds, and 9.95×10⁻⁴ myoblasts per square micrometer for the type I scaffolds. The cellular density in the middle of the type I scaffolds of the present embodiments was 7.26×10⁻⁴ myoblasts per square micrometer.

These results demonstrate that within a predetermined scaffold depth (about 200 μm in the present example), the scaffold of the present embodiments has a cellular density which is higher (by about 23%, in the present example) than the control scaffold.

FIGS. 9C and 9D show a type I scaffold of the present embodiments (FIG. 9C) and a type I control scaffold (FIG. 9D), seeded with hESCs and stained with anti-vimentin (red), phalloidin (green) and DAPl (blue).

The stem cells only poorly penetrated into the type I control scaffold. Almost the entire stem cells population appeared to live on the surface of the control scaffold. FIG. 9D demonstrates that the cellular infiltration into the scaffold is practically negligible.

On the other hand, the stem cells in the type I scaffold of the present embodiments had infiltrated deep into the scaffold (see FIG. 9C). Moreover, due to the high porosity of the scaffold of the present embodiments, the cells were able to create extremely large lumens, with a diameter of over 200 μm.

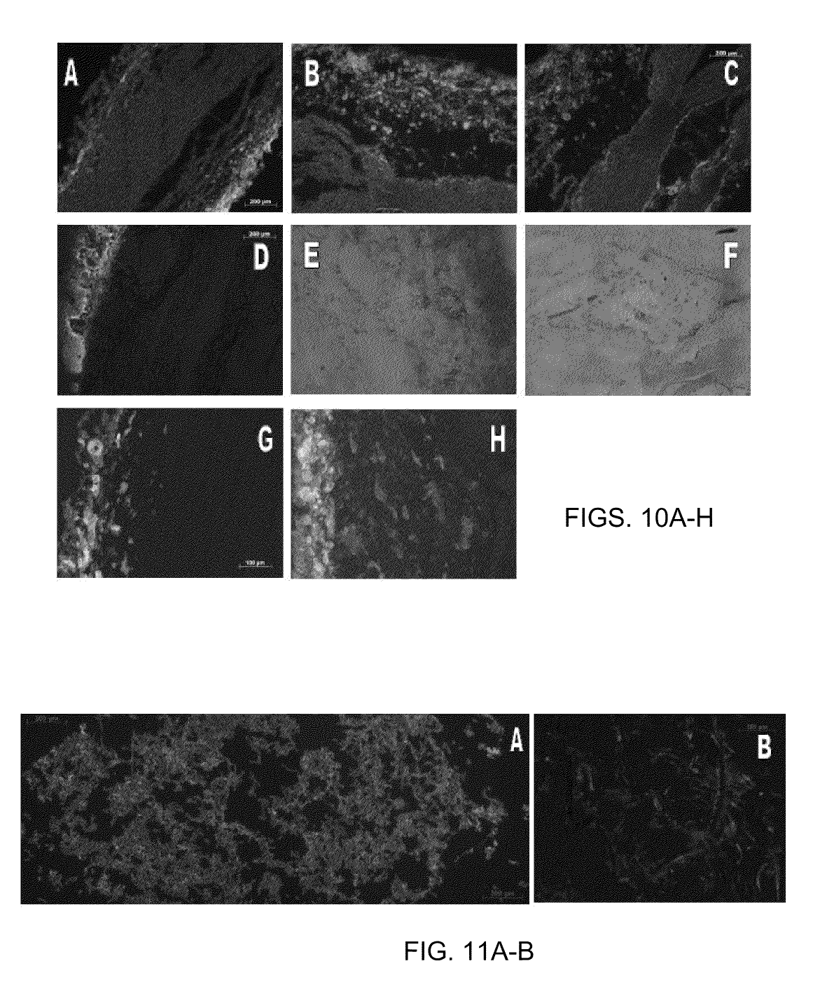

The results of post-spinning seeding technique as employed for type II scaffolds and type II control scaffolds are shown in FIGS. 10A-H.

FIGS. 10A-C show type II control scaffold (FIG. 10A) and type II scaffolds of the present embodiments (FIGS. 10B and 10C) with myoblasts, stained with anti-desmin (red), phalloidin (green) and DAPl (blue).

FIGS. 10D and E show type II control scaffold (FIG. 10D) and type II scaffolds of the present embodiments (FIG. 10E) with hESCs stained with anti-desmin (red), phalloidin (green) and DAPl (blue).

FIG. 10F shows an inner pore inside the type II scaffold of the present embodiments filled with cells.

FIG. 10G shows the type I control scaffold seeded with myoblasts stained with anti-vimentin (red), phalloidin (green) and DAPI (blue);

FIG. 10H shows the type I scaffold seeded with myoblasts stained with anti-vimentin (red), phalloidin (green) and DAPI (blue).

Both the myoblasts and the stem cells did not efficiently infiltrate deeply into the type II control scaffolds. A few individual cells managed to grow as deep as 500 μm into the control scaffold, but the vast majority of the cells were not able to go deeper than 200 μm (see FIGS. 10A and 10D)

On the other hand, in the type II scaffold of the present embodiments, a vastly improved penetration was observed, reaching a depth of 1000 μm at some points. The C2 cells inside the scaffold appeared large and healthy, with rounded nuclei and elongated morphology, as observed by staining for desmin. Both the myoblast and the stem cells were able to reach the large voids between the layers of the scaffold of the present embodiments, and proliferate therein (see FIGS. 10B, 10C, 10E and 10F).

### Cellular Growth Following Layerwise Seeding

The average thickness of the scaffold prepared in this experiment was about 1 mm. FIGS. 11A and 11B show a cross-section of a type I scaffold, 4 days after the scaffold was seeded with myoblasts via the layerwise seeding technique of the present embodiments. Shown in FIG. 11A is the scaffold following staining with DAPl (blue), desmin (red) and FITC-conjugated phalloidin (green). FIG. 11B is an enlarged view of the cells in the scaffold showing the fibers in black.

As shown, the myoblasts have proliferated inside the scaffold, and have remained viable. An equal density of cells was noticeable throughout the scaffold. The results demonstrate that the layerwise seeding technique of the present embodiments allows creating a substantially uniform initial density of cells throughout the three-dimensional scaffold. This technique also allows patterned seeding, to obtain a predetermined three-dimensional organization of the cells in the scaffold.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of fabricating a nonwoven structure, comprising:
forming a nonwoven layer of polymer fibers on a collector liquid surface;
transferring said layer to a solid surface; and
repeating said formation and said transfer in a layerwise manner thereby forming a nonwoven multilayer structure on said solid surface.

2. The method of claim 1, wherein said forming said nonwoven layer is effected by spinning liquefied polymer.

3. The method of claim 2, wherein said liquefied polymer is charged and said spinning is effected by electrostatic forces.

4. The method according to claim 2, wherein said spinning is effected by hydrostatic forces.

5. The method according to claim 1, further comprising drying said multilayer structure.

6. The method of claim 5, wherein said drying comprises vacuum desiccating.

7. The method according to claim 1, wherein said collector liquid is an aqueous liquid.

8. The method according to claims 1, wherein said collector liquid is an organic solvent.

9. The method according to claim 1, wherein said collector liquid comprises saline.

10. The method according to claim 1, further comprising seeding cells on at least one layer of said multilayer structure.

11. The method of claim 10, wherein said seeding is applied to an outermost layer of said multilayer structure.

12. The method of claim 12, wherein said seeding is applied directly to at least one internal layer of said multilayer structure.

13. The method of claim 12, wherein said seeding is applied in a layerwise manner to a plurality of layers of said multilayer structure.

14. The method of claim 12, wherein said seeding is applied such as to form a predetermined density distribution of cells along a thickness direction of said multilayer structure.

15. The method according to claim 1, further comprising incorporating at least one agent in said multilayer structure.

16. The method according to claim 1, wherein said multilayer structure is formed at a rate of at least 20 cubic millimeters per minute.

17. A nonwoven structure, comprising a plurality of nonwoven layers of fibers, wherein an average thickness of the nonwoven structure is at least 0.5 mm, and wherein an overall porosity of the nonwoven structure is at least 90%.

18. The nonwoven structure of claim 17, wherein at least two adjacent layers of said plurality of layers are at least partially spaced apart such said at least two adjacent layers are distinguishable from one another solely by said at least partial spacing.

19. The nonwoven structure according to claim 17, wherein said plurality of layers comprises at least 50 layers.

20. The nonwoven structure according to claim 17, wherein an average thickness per layer is at least 10 times an average fiber diameter.

21. A scaffold for tissue engineering, comprising the nonwoven structure according to claim 17.

22. A cell-scaffold composition, comprising the scaffold of claim 21 and at least one cell population seeded on at least one of said plurality of layers.

23. The composition of claim 22, wherein said cell population has a predetermined density distribution along a thickness direction of said multilayer structure.

24. The composition according to claim 22, wherein an average thickness of said multilayer structure is at least 1 cm and said cell population occupies said multilayer structure at any depth within said multilayer structure.

25. A method of treating a patient, comprising:
implanting in the patient a cell-scaffold composition which comprises a multilayered non woven scaffold and at least one cell population seeded on at least one of said plurality of layers, wherein said scaffold has an average thickness of at least 0.5 mm, and an overall porosity of at least 90%.
